(19) United States
(12) Patent Application Publication
(10) Pub. No.: US 2021/0371598 A1
(43) Pub. Date: Dec. 2, 2021

(54) LINEAR ACETOXY-BEARING SILOXANES AND DESCENDENT PRODUCTS

(71) Applicant: Evonik Operations GmbH, Essen (DE)

(72) Inventors: Wilfried Knott, Essen (DE); Horst Dudzik, Essen (DE); Philippe Favresse, Ratingen (DE)

(73) Assignee: Evonik Operations GmbH, Essen (DE)

(21) Appl. No.: 17/239,011

(22) Filed: Apr. 23, 2021

(30) Foreign Application Priority Data
Jun. 2, 2020 (EP) 20177746.3

(51) Int. Cl. C08G 77/32; C08G 77/14; C08G 77/06

(52) U.S. Cl. CPC C08G 77/32; C08G 77/06; C08G 77/14

(57) ABSTRACT

Described is a process for producing acidified, preferably superacid-acidified, in particular trifluoromethanesulfonic acid-acidified, end-equilibrated linear α,ω-acetoxy-bearing siloxanes, wherein linear α,ω-hydroxy-bearing siloxanes, using acid, preferably superacid, particularly preferably perfluoroalkanesulfonic acid, especially preferably trifluoromethanesulfonic acid, as catalyst, are reacted with acetic anhydride and with addition of acetic acid.

### CROSS REFERENCE TO RELATED APPLICATIONS

This application is a 35 U.S.C. § 119 patent application which claims the benefit of European Application No. 20177746.3 filed Jun. 2, 2020, which is incorporated herein by reference in its entirety.

### FIELD

The invention is in the field of silicone chemistry. It relates to a process for producing linear acetoxy-bearing siloxanes and the use thereof for producing descendent products such as SiOC-based polyether siloxanes.

### BACKGROUND

Routes to acetoxy-functional siloxanes have already been described in the literature. For instance the non-equilibrating opening of simple unbranched siloxane cycles with acetic anhydride to afford short-chain, chain-terminal acetoxy-bearing siloxanes in the presence of catalysts is known.

Borisov and Sviridova describe the opening of cyclic dimethylsiloxanes with acetic anhydride in the presence of catalytic amounts of iron(III) chloride to afford short-chain α,ω-acetoxysiloxanes (S. N. Borisov, N. G. Sviridova, J. Organomet. Chem. 11 (1968), 27-33). Lewis et al. are concerned in U.S. Pat. No. 4,066,680 with the production of short-chain α,ω-siloxanediols, wherein octamethylcyclotetrasiloxane is reacted with acetic anhydride over acid-treated fuller's earths and the thus-obtained mixtures of short-chain α,ω-acetoxysiloxanes are hydrolyzed in alkalified water.

U.S. Pat. No. 3,346,610 likewise discloses a route to acetoxy-bearing, short-chain siloxanes based on metal-halide-induced acetoxy-modification of strained cyclic siloxanes by reacting said siloxanes with acetoxy-containing silicone compounds. A multiplicity of Friedel-Crafts-active metal halides act as a catalyst here, wherein zinc chloride is commended as preferred. A specific objective of U.S. Pat. No. 3,346,610 is the acetoxy-modification of strained diorganosiloxane cycles with deliberate avoidance of equilibration processes.

The prior art thus relates to endeavors which provide for the opening of cyclic siloxanes—here sometimes strained cyclosiloxanes—with acyloxy-containing reactants and which have for their objective to obtain defined linear short-chain siloxane species still requiring separation by means of fractional distillation.

However, the pure-chain acetoxy-modified siloxane compounds of defined molar mass that have been synthesized by this route are unsuitable for production of organomodified siloxanes, in particular polyether siloxanes, that are employed in demanding industrial applications, for example in PU foam stabilization or in the defoaming of fuels, etc. Active ingredients that effectively address such a field of use are always characterized by a broad oligomer distribution comprising high, medium and low molar masses, since the oligomers present therein, depending on their molar mass and hence their diffusion characteristics, can very commonly be imputed to have differentiated surfactant tasks in different time windows of the respective process.

Acyloxyorganopolysiloxanes and here in particular organosiloxanes having terminal acyloxy groups are likewise known as starting materials for subsequent reactions. Thus, for example the acyloxy groups on a diorganosiloxane may be hydrolyzed, whereupon the hydrolysate may be dehydrated and the dehydrated hydrolysate polymerized to form flowable diorganopolysiloxane. These flowable polysiloxanes are suitable as starting materials for production of viscous oils and rubbers which may be cured to afford silicone elastomers.

Organosiloxanes provided with terminal acyloxy groups may be obtained for example by reaction of an alkyl siloxane and an organic acid and/or the anhydride thereof in the presence of sulfuric acid as catalyst. Such a process is described in U.S. Pat. No. 2,910,496 (Bailey et al.). While this process in principle also affords organosiloxanes having terminal acyloxy groups, the process has the disadvantage that the reaction product consists of a mixture of acyloxy-containing siloxanes and acyloxy-bearing silanes of varying composition. In particular, the teaching in this regard explains that alkyl siloxane copolymers composed of M, D and T units are cleaved by the process into trimethylacyloxysilane, diacyloxydimethylsiloxane and methyltriacyloxysilane. Thus, Bailey obtains in the reaction of octamethylcyclotetrasiloxane with acetic anhydride and acetic acid, after neutralization of the sulfuric acid used as catalyst, separation of the salts and removal of water, residual acetic acid and acetic anhydride, a complex substance mixture and certainly not an equilibrate which is then subjected to fractional distillation (see example, ibid.). The chemical identity of the thus obtained fractions II and IV remains unclear and it is therefore difficult in this way to obtain defined products or to separate these in high yields from the mixture.

Citing Bailey et al. (U.S. Pat. No. 2,910,496), DE-OS 1545110 (A1) (Omietanski et al.) teaches a process in which an acyloxy group of an acyloxysiloxane is reacted with the hydroxyl group of a polyoxyalkylenehydroxy polymer to afford a siloxane-oxyalkylene block copolymer and a carboxylic acid, wherein the carboxylic acid is removed from the reaction mixture. The solvent- and catalyst-free reactions described therein in some cases require considerable reaction times (up to 11.5 hours (example 1)), very high reaction temperatures which are harmful to the product (150° C. to 160° C. (example 1)) and application of an auxiliary vacuum/stripping of the reaction matrix with dry nitrogen over the entire reaction duration and despite the harsh reaction conditions do not always achieve complete conversion at the product stage (example 9, ibid.).

From a production engineering standpoint in particular the combination of high reaction temperatures and long reaction times and also the unpredictable product quality are to the detriment of the process described by Omietanski et al.

The teaching of application EP 3611217 A1 discloses that trifluoromethanesulfonic acid-equilibrated α,ω-diacetoxysiloxanes are producible by reaction of siloxane cycles (D₄ and/or D₅) with acetic anhydride in the presence of trifluoromethanesulfonic acid and that these diacetoxysiloxanes react rapidly and also completely with polyether(mono)ols at moderate temperatures to afford SiOC-bonded polyether siloxanes of structure type ABA.

### SUMMARY

The inventors have now surprisingly found that acidified (preferably trifluoromethanesulfonic acid-acidified), end-equilibrated α,ω-diacetoxysiloxanes, especially those described in EP 3611217 A1 and EP 3611216 A1, are also obtainable when linear hydroxy-bearing siloxanes are reacted with acetic anhydride, acid (preferably perfluoroalkanesulfonic acid, in particular trifluoromethanesulfonic acid) and acetic acid.

### DETAILED DESCRIPTION

The present invention therefore provides a process for producing acidified, preferably superacid-acidified, in particular trifluoromethanesulfonic acid-acidified, (end-)equilibrated linear α,ω-acetoxy-bearing siloxanes, wherein
(i) linear α,ω-hydroxy-bearing siloxanes,
(ii) using acid, preferably superacid, particularly preferably perfluoroalkanesulfonic acid, especially preferably trifluoromethanesulfonic acid, as catalyst,
(iii) are reacted with acetic anhydride and with addition of acetic acid.

The acidified, end-equilibrated linear α,ω-diacetoxysiloxanes produced by the process according to the invention and resulting according to the invention have such a high reactivity that they may be further processed for example with polyetherols, polyether diols and/or monools to afford the challenging linear SiOC-bonded polyether siloxane structures and this constitutes further subject matter of the invention.

The corresponding use of the acidified, end-equilibrated linear α,ω-diacetoxysiloxanes resulting according to the invention to produce linear SiOC-bonded polyether siloxanes is therefore further subject matter of the invention.

Acidified (preferably superacid-acidified, in particular trifluoromethanesulfonic acid-acidified) end-equilibrated linear α,ω-diacetoxypolydimethylsiloxanes are obtainable according to the invention when hydroxy-bearing siloxanes, using acid (preferably perfluoroalkanesulfonic acid, in particular trifluoromethanesulfonic acid) as catalyst, are reacted with acetic anhydride and with addition of acetic acid.

The acid (preferably superacid, in particular trifluoromethanesulfonic acid) is by preference employed in amounts of 0.1 to 1.0 percent by weight, preferably 0.1 to 0.3 percent by weight, based on the reaction matrix comprising acetic anhydride and hydroxy-bearing siloxanes. The reaction is preferably carried out in the temperature range from 140° C. to 160° C. and preferably over a period of 4 to 8 hours.

The linear α,ω-hydroxy-bearing siloxanes according to the invention preferably satisfy at least formula (I)

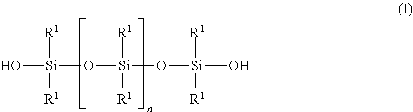

where R¹ is an alkyl radical and/or aromatic radical comprising 1 to 10 carbon atoms, preferably a methyl radical, and where 1≤n≤19 000, preferably n is between 3 and 200, particularly preferably n is between 20 and 100.

According to the invention the terms “acid” and “superacid” are to be understood as meaning Brønsted acids, i.e. proton donor compounds. Contemplated acids/superacids thus include both homogeneous and heterogeneous, both liquid and solid and, among these, particularly also polymeric and/or supported acid systems. Heteropolyacids are also encompassed. Together with acidic hydrogen ions polyoxometallates form heteropolyacids which may be employed as acids in the context of the invention. According to the invention the acids function as catalysts.

In a preferred embodiment of the invention the particularly preferred catalyst superacid, particularly preferably perfluoroalkanesulfonic acid, in particular trifluoromethanesulfonic acid, is employed in amounts of 0.1 to 1.0 percent by weight, preferably 0.1 to 0.3 percent by weight, based on the reaction matrix comprising acetic anhydride and hydroxy-bearing siloxanes.

In a preferred embodiment of the invention acetic acid is added in amounts of 0.4 to 3.5 percent by weight, by preference 0.5 to 3 percent by weight, preferably 0.8 to 1.8 percent by weight, particularly preferably in amounts of 1.0 to 1.5 percent by weight, based on the reaction matrix comprising acetic anhydride and hydroxy-bearing siloxanes.

In a preferred embodiment of the invention the acetic anhydride amount to be employed has to be at least sufficient to ensure that all Si-bonded hydroxy groups of the employed α,ω-hydroxy-bearing siloxane are replaced by acetoxy groups while at the same time the liberated water equivalent is bound in the form of two equivalents of acetic acid by reaction with further acetic anhydride.

In a particularly preferred embodiment the reaction is carried out in a reactor having a volume of at least 1 liter, preferably at least 5 liters, in particular at least 10 liters and preferably not more than 500,000 liters.

The concept of the reactor is known to those skilled in the art. A reactor refers to a delimited space, for example a stirred container or a tube in which chemical transformations of matter may be deliberately performed. As is known to those skilled in the art these may be open or closed containers in which the reactants are converted into the desired products or intermediates. The volume of reactors is specified by the manufacturer or may be determined by volumetric measurement.

In addition to the linear α,ω-hydroxy-bearing siloxanes which are obligatorily to be employed according to the invention, it is optionally also possible to further employ hydroxy-bearing silanes, for example dimethylsilanediol, and/or simple siloxane cycles, in particular comprising D₄ and/or D₅, which may be part of the reaction matrix.

In the context of a preferred embodiment of the invention the acidified, preferably superacid-acidified, in particular trifluoromethanesulfonic acid-acidified, (end-)equilibrated linear α,ω-acetoxy-bearing siloxanes have at least 3, preferably 5 to 50, preferably 7 to 25, particularly preferably 10 to 20, organosiloxane units.

Superacid-acidified, in particular trifluoromethanesulfonic acid-acidified, end-equilibrated linear α,ω-diacetoxypolydimethylsiloxanes are particularly preferred according to the invention.

The term “end-equilibrated” is to be understood as meaning that the equilibrium established at a temperature of 23° C. and a pressure of 1013.25 hPa has been attained. The total cycles content determined by gas chromatography and defined as the sum of the D₄-, D₅-, D₆-contents based on the siloxane matrix and ascertained after derivatization of the α,ω-diacetoxypolydimethylsiloxanes to the corresponding α,ω-diisopropoxypolydimethylsiloxanes may be employed as an indicator for attainment of equilibrium. The inventive use of acetic acid here makes it possible to readily undershoot otherwise customary equilibrium proportions of about 13 percent by weight of the total cycles content for the linear α,ω-diacetoxypolydimethylsiloxanes. It is therefore in accordance with a preferred embodiment when equilibrium proportions of the total cycles content of less than 13, preferably less than 12, percent by weight are undershot for the linear α,ω-diacetoxypolydimethylsiloxanes. The derivatization to afford the α,ω-diisopropoxypolydimethylsiloxanes is intentionally chosen here in order to prevent a thermally induced retrocleavage reaction of the α,ω-diacetoxypolydimethylsiloxanes which may take place under the conditions of analysis by gas chromatography (regarding the retrocleavage reaction see inter alia J. Pola et al., Collect. Czech. Chem. Commun. 1974, 39(5), 1169-1176 and also W. Simmler, Houben-Weyl, Methods of Organic Chemistry, Vol. VI/2, 4th Edition, 0-Metal Derivates of Organic Hydroxy Compounds p. 162 ff.).

As demonstrated the process according to the invention provides the elegant route to acidified, preferably superacid-acidified, in particular trifluoromethanesulfonic acid-acidified, end-equilibrated linear α,ω-acetoxy-bearing siloxanes. The present invention accordingly further provides acidified, preferably superacid-acidified, in particular trifluoromethanesulfonic acid-acidified, end-equilibrated linear α,ω-acetoxy-bearing siloxanes produced by a process as described hereinabove and featuring total cycles contents defined as the sum of the content fractions of the cyclic siloxanes comprising D₄, D₅ and D₆ based on the siloxane matrix and determined by gas chromatography after their derivatization to afford the corresponding linear α,ω-isopropoxysiloxanes of less than 13, preferably less than 12, percent by weight. In a preferred embodiment the acidified, preferably superacid-acidified, in particular trifluoromethanesulfonic acid-acidified, (end-)equilibrated linear α,ω-acetoxy-bearing siloxanes have at least 3, preferably 5 to 50, preferably 7 to 25, particularly preferably 10 to 20, organosiloxane units.

The end-equilibrated acidified, preferably superacid-acidified, in particular trifluoromethanesulfonic acid-acidified, linear acetoxy-bearing siloxanes obtainable according to the invention may be used as starting materials for production of SiOC-bonded linear polyether siloxanes.

The invention accordingly further provides for the use of the end-equilibrated acidified, preferably superacid-acidified, in particular trifluoromethanesulfonic acid-acidified, linear acetoxy-bearing siloxanes as starting materials for production of linear SiOC-bonded polyether siloxanes, in particular for the subsequent use thereof in PU foam stabilizers, in defoamers, in demulsifiers, in emulsifiers and in paint and flow control additives; and also for the use thereof as deaerators; as foam stabilizer, in particular as polyurethane foam stabilizer; as wetting agents; as hydrophobizing agents; as flow control agents; for production of polymer dispersions; for production of adhesives or sealants; for surface treatment of fibers, particles or textile fabrics, in particular for the finishing or impregnation of textiles, for production of paper towels, in the coating of fillers; for production of cleaning and care formulations for household use or for industrial applications, in particular for production of fabric softeners; for production of cosmetic, pharmaceutical and dermatological compositions, in particular cosmetic cleaning and care formulations, hair treatment agents and hair aftertreatment agents; for cleaning and care of hard surfaces; as processing aids in the extrusion of thermoplastics; for production of thermoplastic moulded articles and/or as an adjuvant in plant protection; for production of building material compositions; for production of silicone-containing coatings, in particular silicone release coatings.

In a preferred embodiment of the invention the end-equilibrated linear α,ω-acetoxy-bearing siloxanes are reacted with polyetherols, polyether diols and/or monools, wherein the reaction is carried out in the presence of at least one base, in particular in the presence of carbonate salts, ammonia or an organic amine, and wherein the reaction is preferably carried out in the temperature range from 40° C. to 180° C., preferably between 50° C. and 160° C., particularly preferably between 80° C. and 150° C., to afford the desired SiOC-bonded linear polyether siloxanes.

The replacement of the siloxane-bonded acetoxy groups via the reaction with polyetherols, polyether diols and/or monools may preferably be carried out using an inert solvent, preferably using a solvent which is inert but forms an azeotrope with acetic acid being formed in the reaction or possibly already present, wherein the inert solvent is advantageously an aromatic, preferably alkylaromatic, solvent, and very particularly preferably selected from toluene, xylene and esters selected from methoxypropyl acetate, ethyl acetate or butyl acetate.

In a particularly preferred embodiment the reaction is carried out in a reactor having a volume of at least 1 liter, preferably at least 5 liters, in particular at least 10 liters and preferably not more than 500,000 liters.

In another embodiment the replacement of the siloxane-bonded acetoxy groups via the reaction with polyetherols, polyether diols and/or monools may preferably be carried out solventlessly, i.e. without addition of auxiliary solvents inert in the reaction. By contrast, all compounds having alcoholic OH groups (polyetherols, polyether diols and/or monools) are reactants. According to the invention it is preferable to employ polyetherols of formula (II)

A[-O—(CH₂—CHR′—O—)ₘ-(CH₂—CH₂—O—)ₙ-(CH₂—CH(CH₃)—O-)o-Z]ₐ (II)

where
A is either hydrogen or a saturated or unsaturated organic radical comprising at least one carbon atom, preferably an organic radical, comprising at least one carbon atom, of an organic starter compound for preparing the compound, particularly preferably a methyl, ethyl, propyl, isopropyl, butyl, isobutyl, vinyl or allyl group,
R′ is independently at each occurrence a saturated alkyl group comprising 2-18 carbon atoms or an aromatic radical, preferably an ethyl group or a phenyl radical respectively,
Z is hydrogen,
m=from 0 to 50, preferably from 0 to 30, particularly preferably from 0 to 20,
n=from 0 to 250, preferably from 3 to 220, particularly preferably from 5 to 200,
o=from 0 to 250, preferably from 3 to 220, particularly preferably from 5 to 200,
a=from 1 to 8, preferably from more than 1 to 6, particularly preferably 1, 2, 3 or 4,
   with the proviso that the sum of m, n and o is equal to or greater than 1 and with the proviso that at least A or Z represent hydrogen.

The monools are preferably selected from ethanol, propanol, isopropanol, butanol, isobutanol and polyetherol of formula (II), wherein A then does not represent hydrogen.

In a preferred embodiment at least 1 mol of polyether-bonded OH functionality may be employed per mole of acetoxy group of the siloxane, preferably 1 to 2 mol of polyether-bonded OH functionality, preferably 1.1 to 1.6 mol of polyether-bonded OH functionality, particularly preferably 1.2 to 1.4 mol of polyether-bonded OH functionality, per mole of acetoxy group of the siloxane.

The reaction of the linear α,ω-diacetoxypolydimethylsiloxanes with polyetherols, polyether diols and/or monools is preferably performed in a solvent inert under reaction conditions, wherein preferred solvents are toluene and/or xylenes present in pure form or as an isomer mixture, and wherein these solvents are preferably used in total amounts of 5% to 35% by weight, preferably 10% to 35% by weight, based on the mass of the reaction matrix, and wherein the total water content of the solvents is ≤50 ppm by mass, preferably ≤25 ppm by mass, particularly preferably ≤10 ppm by mass, wherein the determination of the water content is carried out by titration according to Karl Fischer.

The reaction of the linear α,ω-diacetoxypolydimethylsiloxanes with polyetherols, polyether diols and/or monools is preferably performed in the temperature range from 40° C. to 180° C., preferably between 50° C. and 160° C., particularly preferably between 80° C. and 150° C. The reaction of the linear α,ω-diacetoxypolydimethylsiloxanes with polyetherols, polyether diols and/or monools is preferably performed at reduced pressure and/or with passage of an inert gas through the reaction mixture.

It is preferable according to the invention when the acidified (preferably superacid-acidified, in particular trifluoromethanesulfonic acid-acidified) (end-)equilibrated linear α,ω-diacetoxypolydimethylsiloxanes are reacted with polyetherols, polyether diols and/or monools by addition of a solid, liquid or gaseous base, optionally using inert solvents. Preferred simple bases to be employed according to the invention are for example alkali metal and/or alkaline earth metal carbonates and/or hydrogencarbonates and/or gaseous ammonia and/or amines. Given the known tendency to condensation of acetoxysiloxanes, very particular preference is given here to bases that on account of their chemical composition do not introduce any water into the reaction system. Thus anhydrous carbonates are preferred over hydrogencarbonates and bases free from water of hydration are preferred over bases containing water of hydration.

In view of the poor solubility of the alkali metal/alkaline earth metal carbonates and/or hydrogencarbonates in the reaction system a preferred embodiment of the invention comprises choosing relatively high excesses thereof which preferably correspond to at least a 2000-fold stoichiometric equivalent of the acid (preferably superacid, in particular trifluoromethanesulfonic acid) present in the α,ω-diacetoxypolydimethylsiloxane.

According to the invention very particular preference is given to the use of gaseous ammonia as the base so that the acetic acid liberated during the reaction is bound as ammonium acetate.

In a preferred embodiment of the invention the amount of the solid, liquid or gaseous base introduced into the reaction system is measured such that it is sufficient not only for the neutralization of the acid (preferably superacid, in particular trifluoromethanesulfonic acid) present in the system but also for the salt precipitation of the acetate groups bonded to the siloxane and the precipitation of the acetic anhydride and any free acetic acid still present in the reaction system. In a preferred embodiment of the invention the reaction is performed at temperatures between 20° C. and 120° C., preferably between 20° C. and 70° C., over a duration of 1 to 10, preferably at least over the duration of 1 to 3, hours.

A preferred embodiment of the invention may comprise initially charging the acidified (preferably trifluoromethanesulfonic acid-acidified) end-equilibrated linear α,ω-diacetoxypolydimethylsiloxane with polyetherols, polyether diols and/or monools at temperatures of <25° C. with stirring and then adding ammonia. This variant carried out with heavy ammonia addition binds not only acid (preferably superacid, in particular trifluoromethanesulfonic acid), acetic anhydride and any free acetic acid present in the the reaction system but also the acetic acid liberated during the reaction as ammonium acetate. The reaction is preferably carried out at temperatures between 20° C. and 70° C. over a duration of preferably 1 to 3 hours.

Reacting the acidified (preferably trifluoromethanesulfonic acid-acidified), (end-)equilibrated linear α,ω-diacetoxypolydimethylsiloxanes produced according to the invention with polyether diols (formula (II) where A=hydrogen) by addition of a solid, liquid or gaseous base, preferably using a suitable solvent, affords linear A(BA)n-polyether siloxane structures which are in particular of exceptional importance as polyurethane foam stabilizers for viscoelastic PU foams and for so-called mechanically blown PU foam (for example for carpet backing).

Of decisive importance for achieving a high-molecular-weight SiOC-bonded A(BA)n polyether siloxane structure is the quality of the employed acidified (preferably superacid-acidified, in particular trifluoromethanesulfonic acid-acidified) linear α,ω-diacetoxypolydimethylsiloxane. It has been found by the inventors in the context of a broad investigation that, surprisingly, ensuring a perfect equilibration result in the α,ω-diacetoxypolydimethylsiloxane (=end-equilibrated) is essential for the construction of a high-molecular-weight SiOC-bonded A(BA)n polyether siloxane structure.

Obtained in this way in a manner unforeseeable to those skilled in the art are structures which as stabilizers in the production of polyurethane foams (PU foams), in particular flexible PU foams, exhibit markedly better properties.

The invention therefore further provides a process for producing SiOC-bonded, linear polydimethylsiloxane-polyoxyalkylene block copolymers comprising repeating (AB) units by reaction of polyether diols with acidified (preferably trifluoromethanesulfonic acid-acidified) end-equilibrated linear α,ω-diacetoxypolydimethylsiloxanes, wherein the reaction is undertaken by adding a solid, liquid or gaseous base, and optionally using inert solvents.

Equilibrated linear α,ω-diacetoxypolydimethylsiloxanes of this quality, i.e. end-equilibrated α,ω-diacetoxypolydimethylsiloxanes, are producible very advantageously, i.e. also after a very short reaction time, by the reaction of hydroxy-bearing siloxanes with acetic anhydride in the presence of acid (preferably superacid, in particular trifluoromethanesulfonic acid) and acetic acid. It is preferable here when acetic acid is added in amounts of 0.4 to 3.5 percent by weight, preferably 0.5 to 3 percent by weight, more preferably 0.8 to 1.8 percent by weight, particularly preferably in amounts of 1.0 to 1.5 percent by weight, based on the reaction matrix comprising acetic anhydride and hydroxy-bearing siloxanes.

The provision of trifluoromethanesulfonic acid-acidified, end-equilibrated linear α,ω-diacetoxypolydimethylsiloxanes employable according to the invention is for example described in example 1 of the present invention.

A method of reaction monitoring comprising withdrawing over the course of the reaction time samples of the reaction matrix which are then analyzed for example using ²⁹Si-NMR and/or ¹³C-NMR spectroscopy has proven advantageous according to the invention. The reduction in the integral of the signals characteristic of the presence of acetoxydimethylsiloxy groups —OSi(CH₃)₂OCOCH₃ accompanies the intended SiOC-bonding to the desired polyether siloxane copolymer and is a reliable indicator of the reaction conversion achieved.

The linear silicone polyether copolymers produced according to the invention are suitable, alone and/or in admixture with other components, for producing preparations for defoamers, deaerators, foam stabilizers, wetting agents, paint and flow additives or as demulsifiers.

The linear silicone polyether copolymers produced according to the invention are further suitable for production of diesel defoamers, of hydrophobizing agents, of polymer dispersions, of adhesives or sealants, of paper towels; of cleaning and care formulations for the household or for industrial applications, in particular for production of fabric softeners, of cosmetic, pharmaceutical and dermatological compositions, in particular cosmetic cleaning and care formulations, hair treatment agents and hair aftertreatment agents; of construction material compositions, of thermoplastic moulded articles.

Also conceivable is the use of the preparation according to the invention as a processing aid in the extrusion of thermoplastics, as an adjuvant in plant protection, as an additive for the cleaning and care of hard surfaces, for the surface treatment of fibers, particles or fabrics, in particular for the finishing or impregnation of textiles, or in the coating of fillers, as well as for production of silicone-containing coatings, in particular silicone release coatings.

### EXAMPLES

The following examples serve only to explain this invention for those skilled in the art and do not constitute any restriction whatsoever of the claimed subject matter. Determination of the water contents is performed in principle by the Karl Fischer method based on DIN 51777, DGF E-III 10 and DGF C-III 13a. ²⁹Si-NMR spectroscopy was used for reaction monitoring in all examples.

In the context of this invention the ²⁹Si-NMR samples are measured at a measurement frequency of 79.49 MHz in a Bruker Avance III spectrometer equipped with a 287430 sample head with gap width of 10 mm, dissolved at 22° C. in CDCl3 and against a tetramethylsilane (TMS) external standard [δ(²⁹Si)=0.0 ppm].

GPCs (gel permeation chromatography) are recorded using THF as the mobile phase on an SDV 1000/10000A column combination having a length of 65 cm, ID 0.80, at a temperature of 30° C. using a SECcurity² GPC System 1260 (PSS Polymer Standards Service GmbH).

The gas chromatograms are recorded on a GC 7890B GC instrument from Agilent Technologies equipped with an HP-1 column; 30 m×0.32 mm ID×0.25 μm dF (Agilent Technologies No. 19091Z-413E) and hydrogen as carrier gas with the following parameters:

### Detector: FID; 310° C.

Injector: split; 290° C.
Mode: constant flow, 2 ml/min
Temperature programme: 60° C. at 8° C./min-150° C. at 40° C./min 300° C. 10 min.

Employed as an indicator for reaching the equilibrium is the total cycles content determined by gas chromatography and defined as the sum of the D4-, D5-, D6-contents based on the siloxane matrix and ascertained after derivatization of the α,ω-diacetoxypolydimethylsiloxanes to the corresponding α,ω-diisopropoxypolydimethylsiloxanes. The derivatization to afford the α,ω-diisopropoxypolydimethylsiloxanes is intentionally chosen here in order to prevent a thermally induced retrocleavage reaction of the α,ω-diacetoxypolydimethylsiloxanes which may take place under the conditions of analysis by gas chromatography (regarding the retrocleavage reaction see inter alia J. Pola et al., Collect. Czech. Chem. Commun. 1974, 39(5), 1169-1176 and also W. Simmler, Houben-Weyl, Methods of Organic Chemistry, Vol. VI/2, 4th Edition, O-Metal Derivates of Organic Hydroxy Compounds p. 162 ff.).

### Example 1 (Inventive)

Production of an Acetoxy-Terminated, Linear Polydimethylsiloxane with 3.0% Acetic Acid Addition

In a 1000 ml four-necked flask with a KPG stirrer, internal thermometer and fitted with a reflux cooler 77.3 g (0.757 mol) of acetic anhydride together with 732.8 g (0.267 mol) of an α,ω-dihydroxypolydimethylsiloxane (M: 2742 g/mol and 24.3 g of acetic acid (3.0% by weight based on the total mass of the reactants) are initially charged with stirring and admixed with 1.62 g (0.88 ml) of trifluoromethanesulfonic acid (0.2 percent by weight based on the total batch) and rapidly heated to 150° C. The initially slightly cloudy reaction mixture is left at this temperature for 4 hours with continued stirring.

After cooling the mixture, a colorless, clear, mobile liquid is isolated, the ²⁹Si-NMR spectrum of which demonstrates the presence of Si-acetoxy groups in a yield of approx. 93% based on acetic anhydride used, corresponding to an α,ω-diacetoxypolydimethylsiloxane having an average total chain length of approx. 14.

Conversion of the linear α,ω-diacetoxypolydimethylsiloxane into the corresponding α,ω-dii sopropoxypolydimethylsiloxane for analytical characterization

Immediately after the synthesis, in a 250 ml four-necked round-bottomed flask fitted with a KPG stirrer, internal thermometer and fitted with a reflux cooler 50.0 g of this trifluoromethanesulfonic acid-acidified, equilibrated α,ω-diacetoxypolydimethylsiloxane are mixed together with 11.3 g of a molecular sieve-dried isopropanol with stirring at 22° C. Gaseous ammonia (NH₃) is then introduced to the reaction mixture until alkaline reaction (moist universal indicator paper) and the mixture is then stirred at this temperature for a further 45 minutes. The precipitated salts are removed using a fluted filter.

A colorless, clear liquid is isolated, whose accompanying ²⁹Si-NMR spectrum demonstrates the quantitative conversion of the α,ω-diacetoxypolydimethylsiloxane into an α,ω-diisopropoxypolydimethylsiloxane.

An aliquot of this α,ω-diisopropoxypolydimethylsiloxane is withdrawn and analyzed by gas chromatography. The gas chromatogram shows the following contents (reported in percent by weight):

| | | | Sum of | Isopropanol |
|---|---|---|---|---|
| D₄ | D₅ | D₆ | (D₄-D₆) | content |
| 4.12% | 2.61% | 0.84% | 7.57% | 4.58% |

Taking account of the isopropanol excess the contents of siloxane cycles (D₄, D₅ and D₆) are calculated here solely based on the siloxane proportion.

1. A process for producing acidified, preferably superacid-acidified, in particular trifluoromethanesulfonic acid-acidified, (end-)equilibrated linear α,ω-acetoxy-bearing siloxanes, wherein
(i) linear α,ω-hydroxy-bearing siloxanes,
(ii) using acid as catalyst,
(iii) are reacted with acetic anhydride and with addition of acetic acid.

2. The process according to claim 1, wherein the acid employed in addition to the acetic acid is a superacid having a pKa of less than −3.0.

3. The process according to claim 1, wherein the linear α,ω-hydroxy-bearing siloxanes satisfy formula (I): 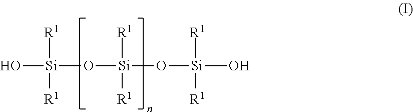
where R¹ is independently at each occurrence an alkyl radical and/or aromatic radical comprising 1 to 10 carbon atoms.

4. The process according to claim 1, wherein the acetic acid is added in amounts of from 0.4 to 3.5 percent by weight based on the reaction matrix comprising acetic anhydride and hydroxy-bearing siloxanes, and in that in addition to the acetic acid further acid, is employed in amounts of from 0.1 to 1.0 percent by weight, based on the reaction matrix comprising acetic anhydride and hydroxy-bearing siloxanes.

5. The process according to claim 1, wherein the acetic anhydride amount to be employed has to be at least sufficient to ensure that all Si-bonded hydroxy groups of the employed α,ω-hydroxy-bearing siloxane are replaced by acetoxy groups while at the same time the liberated water equivalent is bound in the form of two equivalents of acetic acid by reaction with further acetic anhydride.

6. The process according to claim 1, wherein the reaction is carried out in a reactor having a volume of at least 1 liter.

7. The process according to claim 1, wherein the acidified, preferably superacid-acidified, in particular trifluoromethanesulfonic acid-acidified, (end-)equilibrated linear α,ω-acetoxy-bearing siloxanes have at least 3, organosiloxane units.

8. An acidified, (end-)equilibrated linear α,ω-acetoxy-bearing siloxanes produced by a process according to claim 1, wherein the acidified (end-)equilibrated linear α,ω-acetoxy-bearing siloxanes have total cycles contents defined as the sum of the content fractions of the cyclic siloxanes comprising D₄, D₅ and D₆ based on the siloxane matrix and determined by gas chromatography after their derivatization to afford the corresponding linear α,ω-isopropoxysiloxanes of less than 13 percent by weight, and wherein (end-)equilibrated linear α,ω-acetoxy-bearing siloxanes have at least 3 organosiloxane units.

9. A process for making SiOC-bonded linear polyether siloxanes comprising using the (end-)equilibrated acidified acetoxy-bearing siloxanes according to claim 8 as starting materials for production of SiOC-bonded linear polyether siloxanes.

10. The process according to claim 9 by reaction of the (end-)equilibrated linear α,ω-acetoxy-bearing siloxanes with polyetherols, polyether diols and/or monools, wherein the reaction is carried out in the presence of at least one base, and wherein the reaction is carried out in the temperature range from 40° C. to 180° C.

11. The process according to claim 10, wherein the reaction is carried out using an inert solvent.

12. The process according to claim 10, wherein the polyetherols are those of formula (II)
A[-O—(CH₂—CHR′—O—)ₘ-(CH₂—CH₂—O—)ₙ-(CH₂—CH(CH₃)—O—)ₒ-Z]ₐ (II)
wherein
A is either hydrogen or a saturated or unsaturated organic radical comprising at least one carbon atom,
R′ is independently at each occurrence a saturated alkyl group comprising 2-18 carbon atoms or an aromatic radical,
Z is hydrogen,
m=from 0 to 50,
n=from 0 to 250,
o=from 0 to 250,
a=from 1 to 8,
wherein the sum of m, n and o is equal to or greater than 1 and
wherein at least A or Z represent hydrogen.

13. The process according to claim 10, wherein the monools are selected from ethanol, propanol, isopropanol, butanol, isobutanol and polyetherol of formula (II).

14. The process according to claim 10, wherein at least 1 mol of polyether-bonded OH functionality is employed per mole of acetoxy group of the siloxane per mole of acetoxy group of the siloxane.

15. The process according to claim 10, wherein the reaction of the acetoxy-bearing siloxane with polyetherol is performed in a solvent inert under reaction conditions, and wherein these solvents are used in total amounts of from 5% to 35% by weight, based on the mass of the reaction matrix, and wherein the total water content of the solvents is ≤50 ppm by mass, wherein the determination of the water content is carried out by titration according to Karl Fischer.

16. The process according to claim 10, wherein the reaction is carried out in a reactor having a volume of at least 1 liter.

17. The preparation obtained according to claim 10 containing at least one SiOC-bonded, linear silicone polyether, a polyetherol and an acetyl-endcapped polyether wherein the polyether radical present in the silicone polyether is chemically identical to the polyether radical of the polyetherol and to the polyether radical of the acetyl-endcapped polyether and that the proportion of the SiOC-bonded, linear silicone polyether is at least 50 percent by weight based on the overall preparation, and the use thereof
(a) for production of defoamers, deaerating agents, foam stabilizers, wetting agents, paint and flow control additives or as demulsifiers,
(b) for production of diesel defoamers, of hydrophobizing agents, of polymer dispersions, of adhesives or sealants, of paper towels; of cleaning and care formulations for the household or for industrial applications, in particular for production of fabric softeners, of cosmetic, pharmaceutical and dermatological compositions, in particular cosmetic cleaning and care formulations, hair treatment agents and hair aftertreatment agents; of construction material compositions, of thermoplastic moulded articles,
or
(c) as a processing aid in the extrusion of thermoplastics, as an adjuvant in plant protection, as an additive for the cleaning and care of hard surfaces, for the surface treatment of fibers, particles or fabrics, in particular for the finishing or impregnation of textiles, or in the coating of fillers
or
(d) for production of silicone-containing coatings, in particular silicone release coatings.

18. The process according to claim 1, wherein the acid is a fluorinated and/or perfluorinated sulfonic acid.

19. The process according to claim 1, wherein the acid is selected from the group consisting of fluorosulfonic acid, fluoroantimonic acid, perfluorobutanesulfonic acid, and trifluoromethanesulfonic acid.

20. The process according to claim 1, wherein the acetic acid is added in amounts of from 0.8 to 1.8 percent by weight based on the reaction matrix comprising acetic anhydride and hydroxy-bearing siloxanes, and in that in addition to the acetic acid trifluoromethanesulfonic acid, is employed in amounts of from 0.1 to 0.3 percent by weight based on the reaction matrix comprising acetic anhydride and hydroxy-bearing siloxanes.
